SEQUENCE LISTING (3 sequences)

SEQ ID NO: 1 - protein, 514 aa, Homo Sapiens
MAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIRVPMASCDFSIRTYTYADTPDDFQLHNFSLPEEDTKLKIPLHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - protein, 232 aa, Homo Sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 228 aa, Homo Sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2004/0043457 A1
(43) Pub. Date: Mar. 4, 2004

(54) Bifunctional fusion proteins with glucocerebrosidase activity

(76) Inventors: Silke Schumacher, Heidelberg (DE); Stephen Gillies, Carlisle, MA (US)

Correspondence Address: OLSON & HIERL, LTD., 20 NORTH WACKER DRIVE, 36TH FLOOR, CHICAGO, IL, 60606, US

(21) Appl. No.: 10/466,593

(22) PCT Filed: Dec. 27, 2001
(86) PCT No.: PCT/EP01/15328

(30) Foreign Application Priority Data
Jan. 18, 2001 (EP) 01101056.8

(51) Int. Cl. C12P 21/04; C07K 16/46; C12N 5/06

(52) U.S. Cl. 435/69.7; 530/391.1; 435/326; 435/320.1

(57) ABSTRACT

The present invention relates to novel Glucocerebrosidase bifunctional fusion proteins consisting essentially of an Immunoglobulin (Ig) molecule and a protein having the biological activity of Glucocerebrosidase, for enzyme replacement therapy and/or augmentation of glycolipid metabolism by the administration of bifunctional fusion proteins using a therapy based on the treatment of glycolipid storage disorders such as Gaucher's, Fabry's and Tay-Sachs diseases.

### FIELD OF THE INVENTION

The present invention relates to Glucocerebrosidase (GCR) bifunctional fusion proteins (GCR fusion proteins) consisting essentially of an Immunoglobulin (Ig) molecule (whole antibody, an Ig heavy or light chain or a fragment thereof and a protein (the term includes also oligopeptides) having the biological activity of GCR (GCR-like protein), for enzyme replacement therapy and/or augmentation of glycolipid metabolism by the administration of bifunctional fusion proteins using a therapy based on the treatment of glycolipid storage disorders such as Gaucher's, Fabry's and Tay-Sachs diseases.

By selective altering of the amino acid sequences of the Ig moiety, GCR fusion proteins with improved properties, e.g. enhanced stability, can be obtained. Furthermore, fusion proteins can be provided, wherein shortened versions of GCR and the Ig chain are used.

The present invention relates also to pharmaceutical compositions and therapeutic methods and systems comprising such GCR fusion proteins and methods of treating Gaucher's disease or another disease caused by glycolipid storage disorders, such as Fabry's and Tay-Sachs disease, comprising administering to a subject afflicted with this disease, a pharmaceutical composition comprising a therapeutic amount of recombinantly produced GCR fusion protein in a pharmaceutically acceptable carrier.

### BACKGROUND

The administration of exogenous β-glucosidase to treat diseases caused by glycolipid storage disorders like Gaucher's, Tay-Sachs' or Fabry's disease as attempts of enzyme augmentation in an organism suffering from such a disease rather than splenectomy or bone marrow transplantation are already described in literature to treat lysosomal storage defects. See, for example, De Duve, C. in Fed. Proc. 23, 1045 (1964) and Barton, N. W. et al. in Proc. Natl. Acad. Sci. 87, 1913 (199) which describe the use of β-glilcosidases and especially GCR to treat Gaucher's disease and the difficulties combined therewith to get a therapeutic response. However, the dose of the enzyme to treat these diseases is about 60 units per kilogram body weight every two weeks, that means that the average costs per year for the treatment of a 70 kg patient are about US$ 380.000,—for the enzyme alone. This is due to the short intracellular half-life of exogenous acid β-glucosidase.

Antibody-enzyme fusion proteins have been described which have a considerable improved in-vivo half-life and promote targeting to specific cell types such as tumor cells. For example the cytokine interleukin 2 (IL-2) has been fused to a monoclonal antibody heavy chain immunoreactive with, in two separate fusion proteins, the tumor antigens epithelial cell adhesion molecule (Ep-CAM) or the disialoganglioside GD2 by use of the antibodies KS1/4 and ch14.18, respectively, to form the fusion proteins ch14.18-IL-2 and KS1/4-IL-2, respectively. See, for example, U.S. Pat. No. 5,650,150.

Therefore, the object of the invention was to find suitable compounds for the effective treatment of glycolipid storage disorders, such as Gaucher's, Fabry's and Tay-Sachs disease which allow an efficient way and mode of administration, which is cheaper than the known costly procedures and within the price range of most patients, especially those in developing countries. The goal of the invention was to provide molecules for the treatment of Gaucher's, Fabry's and Tay-Sachs disease which can be administered in low dosages and have a longer half life in an organism without a significantly reduced activity and better targeting to specific cells where the glycolipid metabolism takes place and therefore enable a cheaper and more effective treatment of these diseases.

### SUMMARY OF THE INVENTION

It has now been discovered that there is an unexpected synergy, in effectiveness and a prolonged half-life, if proteins having the biological activity of GCR are linked to an Ig molecule like an whole antibody, an Ig heavy or light chain, a fragment of an Ig heavy chain, for example the constant region of the heavy chain (C_{H}), or the Fc or Fab fragment.

Fusion proteins and modification of specified fusion proteins are known in the art. For example, fusion proteins may effectively block a proteolytic enzyme from physical contact with the protein backbone itself, and thus prevent degradation. Additional advantages include, under certain circumstances, improved yield in a specific expression system, correct folding of a target protein, and increasing the stability, circulation time, and the biological activity of the therapeutic protein. One such modification is the use of the Fc region of immunoglobulins. Antibodies comprise two functionally independent parts, a variable domain known as “Fab”, which binds antigen, and a constant domain, known as “Fc” which provides the link to effector functions such as complement or phagocytic cells.

The Fc portion of an immunoglobulin mediates a long plasma half life when fused to certain proteins that have particularly short half lives (Capon, et al.,Nature 337: 525-531 (1989)).

Therapeutic fusion proteins have also been constructed using the Fc domain to incorporate functions such as Fc receptor binding, protein A binding, complement fixation and placental transfer which all reside in the Fc proteins of immunoglobulins. For example, the Fc region of an IgG1 antibody has been fused to the N-terminal end of CD30-L, a molecule which binds CD30 receptors expressed on Hodgkin's Disease tumor cells, anaplastic lymphoma cells, T-cell leukemia cells and other malignant cell types (U.S. Pat. No. 5,480,981). Furthermore, it has been reported in 1996 that efficient expression and secretion of certain non-mutant target proteins can be achieved by expression of fusion proteins comprising an Fc portion of an immunoglobulin and said target proteins followed by proteolytic cleavage of the target protein (WO 96108570, U.S. Pat. No. 5,541,087).

A suitable GCR-like protein to be fused with an Ig polypeptide chain can have an amino acid sequence and a relating DNA sequence as given in the U.S. Pat. No. 5,879,680 or can be a truncated or mutated form derived therefrom. Exemplary these proteins and the method of synthesis and conditions thereof, excluding the truncated and mutated forms, are described in the teachings of U.S. Pat. No. 5,879,680, the disclosures of which relating to the preparation and use are specifically incorporated herein by reference.

Preferred truncated forms are for example those which consist of about one third to one half of the amino acid sequence of the natural GCR enzyme truncated from the carboxy terminal site of the enzyme. Those truncated proteins may be derived from the full length protein by cleaving off the desired chain with a suitable reagent such as a restriction enzyme or the like.

Assays for the identification of an effective GCR-like protein which is a suitable candidate for the fusion with an Ig polypeptide chain, as well as for the proof of activity for a fused compound according to this invention are described in the referenced U.S. Patent, and therefore it is considered that alternate fusion proteins for the treatment of glycolipid storage disorders such as Gaucher's, Fabry's and Tay-Sachs disease can be readily identified for practicing the present invention.

The invention presents novel proteins that have GCR-like activity in their ability to hydrolyze glucocerebrosides in an animal, but with additional advantageous properties such as higher expression level, higher solubility, better tissue distribution and better targeting to macrophages. These novel proteins include fusion proteins of GCR-like proteins and Ig molecules like a whole antibody or fragments thereof (an Ig heavy or light chain or a fragment of the heavy chain, like the C_{H}, Fc or Fab fragment), forms of these fusion proteins that have altered glycosylation either in the GCR-like protein or in the Ig portion, forms of GCR fusion proteins that have a truncated or mutated amino acid sequence, having, for example, a reduced affinity e.g. to neonatal Fc receptors (FcRn) and GCR fusion proteins having specific linkers.

### DETAILED DESCRIPTION

It is an object of the present invention to provide a protein with GCR-like activity having improved properties, wherein said protein is a fusion protein comprising an Ig molecule like a whole antibody, an Ig heavy or light chain or a fragment of the heavy chain (e.g. the C_{H}, Fc or Fab fragment) and an GCR-like protein, wherein said Ig moiety is fused covalently directly or indirectly (via a linker molecule) to said GCR-like protein. In a preferred embodiment, the Ig moiety is fused covalently via its C-terminus directly or indirectly (via a linker molecule) to said GCR-like protein by its N-terminus, and the Ig portion as well as the GCR portion may be modified or mutated, selected from the group:

(I) H₂N-Ig—GCR-COOH

(II) H₂N-Ig—L—GCR-COOH

(III) H₂N-Ig—GCRₘ-COOH

(IV) H₂N-Igₘ—GCR-COOH

(V) H₂N-Igₘ—GCRₘ-COOH

(VI) H₂N-Igₘ—L—GCR-COOH

(VII) H₂N-Ig—L—GCRₘ-COOH

(VIII) H₂N-Ig—GCR_{trunc}-COOH

(IX) H₂N-Ig—L—GCR_{trunc}-COOH

(X) H₂N-GCR—Ig-COOH

(XI) H₂N-GCR—L—Ig-COOH

(XII) H₂N-GCRₘ—Ig-COOH

(XIII) H₂N-GCR—Igₘ-COOH

(XIV) H₂N-GCRₘ—Igₘ-COOH

(XV) H₂N-GCR—L—Igₘ-COOH

(XVI) H₂N-GCRₘ—L—Ig-COOH

(XVII) H₂N-GCR_{trunc}—Ig-COOH

(XVIII) H₂N-GCR_{trunc}—L—Ig-COOH

Herein, Ig has the meaning of a Ig heavy or light chain or a fragment of an Ig heavy chain (e.g. the C_{H}, Fc or FAB fragment). GCR has the meaning of naturally occurring GCR from mammalian, preferably human origin, especially preferred from human lysosomal origin, and includes also recombinant GCR engineered from natural sources.

GCR_{trunc} is an GCR according to this invention which is truncated but not mutated in its amino acid sequence. Truncated forms are protein fragments having essentially the full or only a slightly reduced biological activity of glucocerebrosidase. Preferred truncated forms of GCR according to this invention are those which consist of about one third to one half of the amino acid sequence of the natural glucocerebrosidase enzyme shortened at the C-terminus.

GCRₘ is an GCR according to this invention which is mutated but not truncated in its amino acid sequence. The number of mutations is not limited but is restricted to the loss of the biological activity of the molecule. In a preferred embodiment the degree of mutation is between 5 and 30 per cent, in a especially preferred embodiment between 5 and 20 per cent of the amino acid residues. Variants with increased GCR biological activity can be generated by procedures described known in the art.

GCR, GCRₘ, GCR_{trunc} according to the invention is glycosylated, non-glycosylated, partially glycosylated or otherwise modified in its glycosylation pattern.

The GCR fusion protein can be purified by standard techniques, for example, on a protein A column.

L has the meaning of a series of peptides such as. e.g., glycine and/or serine. Preferably, the peptide linker is a mixed series of glycine and serine peptides about 5-25, preferably 10-20 residues in length. Especially preferred are proteolytically cleavable linkers, especially linkers which are cleavable by lysosomal proteases like cathepsins.

In a preferred embodiment the Ig moiety is specific for a cell bearing an Fc receptor. Therefor, a preferred fragment of an Ig molecule to be linked to GCR is the Fc region. Th Fc region of an immunoglobulin is the amino acid sequence for the carboxyl-terminal portion of an immunoglobulin heavy chain constant region. The Fc regions are particularly important in determining the biological functions of the immunoglobulin and these biological functions are termed effector functions. As known, the heavy chains of the immunoglobulin subclasses comprise four or five domains: IgM and IgE have five heavy chain domains, and IgA, IgD and IgG have four heavy chain domains. The Fc region of IgA, IgD and IgG is a dimer of the hinge-CH₂—CH₃ domains, and in IgM and IgE it is a dimer of the hinge-CH₂—CH₃—CH₄ domains (see, W. E. Paul, ed., 1993, Fundamental Immunology, Raven Press, New York, N.Y.).

As used herein, the term “Fc portion” means the carboxyl-terminal portion of an immunoglobulin heavy chain constant region, or an analog or portion thereof. That is, e.g., an immunoglobulin Fc region of Ig, preferably IgG, which may comprise at least a portion of a hinge region, a CH2 domain, and a CH3 domain.

The Fc region can be joined at its amino-terminus by a peptide bond to the carboxy-terminal amino acid of the GCR, or, in a preferred embodiment, the Fc region is linked at its carboxy-terminus by a peptide bond to the amino-terminal amino acid of the GCR.

In some circumstances, it is useful to mutate certain amino acids within the Ig molecule, especially in the Fc region of the fusion protein. For example, the neonatal Fc receptor (FcRn) binds IgG, and might reduce the clinical efficacy of the fusion protein.

Thus, Fcₘ is a Fc portion as defined above which is mutated in its amino acid sequence and/or modified in its glycosylation pattern. Such modified Fc portions lead to fusion proteins with improved properties. In this context Fcₘ includes additionally modified or mutated Fc portions which have a reduced affinity to FcRn receptors. For example, it is known that IgG histidins located at the junction between the CH2 and CH3 domains (residues 310 and 433) of the IgG heavy chain contribute to the pH-dependent binding to the FcRn receptor (Raghavan, et al.,Biochemistry 34(45): 14649-57 (1995)). Also IIe 253 and His 435 and 436 (Kim et al., Eur. J. Immunol. 34: 2429-34 (1994)) as well as residues 309 (Leu, Val, Gln or Met in rat, murine and human IgGs) and 311 (Gln or Arg in rat, murine and human IgGs) (Kabat et al., in: Sequences of proteins of immunological interest. US Department of Health and Human Services, Bethesda, Md., USA (1991)) seems to form an interaction with the FcRn receptor. Thus, it is an object of the invention to provide a fusion protein with enhanced in vivo circulating half-life having a mutation, deletion or insertion at one or more amino acids in the domains responsible for FcRn receptor binding.

In a preferred embodiment of the invention the GCR fusion protein comprises a Fc portion of an IgG1, wherein said mutations are: position 253 is not IIe, position 309 is not Leu, Val, Gln or Met, position 310 is not His, position 311 is not Gln or Arg, position 433 is not His, position 435 is not His, and position 436 is not His. These and other variant proteins according to the invention may establish enhanced binding to the Fc receptor, enhanced stability, enhanced adoption of a correct active conformation, enhanced pharmacokinetic properties, enhanced synthesis, or other advantageous features. A specific method for improvement of GCR fusion proteins uses site-directed mutagenesis techniques. It is important to note that a wide variety of site-directed mutagenesis techniques are available, and can be used as alternatives to achieve similar results. The strategies for choosing among these techniques is well-known to those skilled in the art of molecular biology. Similarly, there is a wide variety of techniques for achieving random and semi-random mutagenesis of a target DNA. These techniques are also well-known to those skilled in the art of molecular biology.

The Ig molecule and the GCR-like protein according to this invention may also be linked by linker molecules, wherein the amino acid linkers are of varying length. The linker of the invention (L) is a linker molecule as defined below which may have also a protease cleavage site.

The peptide linker often is a series of peptides such as. e.g., glycine and/or serine. Preferably, the peptide linker is a mixed series of glycine and serine peptides about 5-25, preferably 10-20 residues in length. Especially preferred are proteolytically cleavable linkers, especially linkers which are cleavable by lysosomal proteases like cathepsins.

Preferred amino acid linkers L are used and include the following sequences:

1. Ala Ala Ala

2. Ala Ala Ala Ala,

3. Ala Ala Ala Ala Ala,

4. Ser,

5. Ser Ser,

6. Gly Gly Gly,

7. Gly Gly Gly Gly,

8. Gly Gly Gly Gly Gly,

9. Gly Gly Gly Gly Gly Gly Gly,

10. Gly Pro Gly,

11. Gly Gly Pro Gly Gly,

12. Gly Gly Gly Gly Ser, and, if the linker shall have a protease cleavage site

13. Gly Gly Tyr Leu

14. Gly Gly Tyr

15. Gly Phe Ala Leu

16. Gly Pro Arg Leu and

17. any combinations of subparts 1-16

Additional suitable linkers are disclosed in Robinson et al., 1998, Proc. Natl. Acad. Sci. USA; 95, 5929.

As used herein, “proteolytic cleavage site” means amino acid sequences which are preferentially cleaved by a proteolytic enzyme or other proteolytic cleavage agents. Proteolytic cleavage sites include amino acids sequences which are recognized by proteolytic enzymes especially cathepsins or other lysosomal proteases.

It is another object of the present invention to construct GCR fusion proteins, wherein a whole antibody is used. Such fusion molecules comprise the variable regions of heavy and light chains of an antibody and the epitopes binding to a specific antigen. For example, GCR is fused to the C-terminus of an antibody heavy chain. DNA constructs encoding whole antibody fusion proteins may be constructed as described previously (Gillies et al. [1991] Hybridoma 10:347-356).

The invention also relates to a DNA molecule that encodes any of the fusion proteins disclosed above and depicted in the claims.

As a preferred embodiment a DNA molecule is disclosed that encodes a fusion protein as defined above and in the claims comprising:

(a) a signal/leader sequence

(b) a sequence of an Ig molecule

(c) a target protein sequence having the biological activity of GCR.

The signal sequence of the invention as indicated above is a polynucleotide which encodes an amino acid sequence that initiates transport of a protein across the membrane of the endoplasmic reticulum. Signal sequences which will be useful in the invention include antibody light chain signal sequences, e.g., antibody 14.18 (Gillies et. al., Jour. of Immunol. Meth., 125:191, (1989)), antibody heavy chain signal sequences, e.g., the MOPC141 antibody heavy chain signal sequence (Sakano et al., Nature 286:5774(1980)), and any other signal sequences which are known in the art (see for example, Watson, Nucleic Acids Research 12:5145, (1984)). Each of these references is incorporated herein by reference. Signal sequences have been well characterised in the art and are known typically to contain 16 to 30 amino acid residues, and may contain greater or fewer amino acid residues. A typical signal peptide consists of three regions: a basic N-terminal region, a central hydrophobic region, and a more polar C-terminal region. The central hydrophobic region contains 4 to 12 hydrophobic residues that anchor the signal peptide across the membrane lipid bilayer during transport of the nascent polypeptide. Following initiation, the signal peptide is usually cleaved within the lumen of the endoplasmic reticulum by cellular enzymes known as signal peptidases.

Potential cleavage sites of the signal peptide generally follow the “(−3, −1) rule”. Thus a typical signal peptide has small, neutral amino acid residues in positions −1 and −3 and lacks proline residues in this region. The signal peptidase will cleave such a signal peptide between the −1 and +1 amino acids. Thus, the portion of the DNA encoding the signal sequence may be cleaved from the amino-terminus of the fusion protein during secretion. This results in the secretion of a fusion protein consisting of the Ig region and the target protein. A detailed discussion of signal peptide sequences is provided by von Heijne (Nucleic Acids Res., 14:4683,(1986)). As would be apparent to one of skilled in the art, the suitability of a particular signal sequence for use in a secretion cassette may require some routine experimentation. A signal sequence is also referred to as a “signal peptide”, “leader sequence” or “leader peptides” and each of these terms having meanings synonymous to signal sequence may be used herein.

The invention also relates to expression vectors comprising said DNA molecules which promote expression of the target protein, that is a GCR fusion protein. As used herein, “vector” means any nucleic acid comprising a nucleotide sequence competent to be incorporated into a host cell and to be recombined with and integrated into the host cell genome, or to replicate autonomously as an episome. Such vectors include linear nucleic acids, plasmids, phagemids, cosmids, RNA vectors, viral vectors and the like. Non-limiting examples of a viral vector include a retrovirus, an adenovirus and an adeno-associated virus. As used herein, “expression of a target protein” is understood to mean the transcription of the DNA sequence, translation of the mRNA transcript, and secretion of a protein product that is folded into a correct, active conformation.

According to the invention eukaryotic, preferably mammalian, host cells are used that are suitable for expressing a fusion protein as defined in this application. Methods of transfecting such host cells with said vector, expressing, purifying and isolating the fusion proteins of this invention are well known in the art. Therefore, the method according to this invention comprises:

(i) constructing a DNA encoding a precursor protein that comprises a leader sequence for secretion, the Ig portion, the GCR, GCRₘ or GCR_{trunc} moiety and optionally a linker sequence between the Ig and GCR portion.

(ii) placing said fused DNA in an approbiate expression vector,

(iii) expressing said fusion protein in a eukaryotic cell, and

(iv) purifying said secreted fusion protein.

The invention also relates to pharmaceutical compositions comprising at least one of the GCR fusion protein as defined above and below, preferably a fusion protein wherein a Fc portion of a IgG is linked at its C-terminal amino acid by a peptide bond to the N-terminal amino acid of the GCR-like protein, together with pharmaceutically acceptable carriers, diluents, and excipients. These pharmaceutical compositions may optionally contain other drugs or medicaments that are helpful in co-treating GCR deficient diseases.

Such pharmaceutical compositions may be for intravenous, subcutaneous, intramuscular, orthotopic injection, orthotopic infusion, or for oral, pulmonary, nasal, transdermal or other forms of administration. Administration can be accomplished by periodic unit dosages, by continuous infusion, peristaltic delivery, by bolus injection, and the like. Routes can include

In general, comprehended by the invention are pharmaceutical compositions comprising effective amounts of protein or derivative products of the invention together with pharmaceutically acceptable diluents, preservatives, solubilizers, emulsifiers, adjuvants and/or carriers. Such compositions include diluents of various buffer content (e.g., Tris-HC1, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The term “parenteral” as mentioned above and below includes subcutaneous, intravenous, intra-articular and intratracheal injection and infusion techniques. The parenteral administration is preferred.

As used herein, the term “pharmaceutically acceptable carrier or excipient” means an inert, non toxic liquid filler, diluent, solvent or solution, not reacting adversely with the active compounds or with the patient. Suitable liquid carriers are well known in the art such as steril water, saline, aqu ous dextrose, sugar solutions, ethanol, glycols and oils, including those of petroleum, animal, vegetable, or synthetic origin. The formulations may also contain adjuvants or vehicles which are typical for parenteral administration.

With respect to said suitable formulations it should be pointed out that the Fusion proteins of the present invention may eventually form pharmaceutically acceptable salts with any non-toxic, organic or inorganic acid showing changed solubility. Inorganic acids are, for example, hydrochloric, sulphuric or phosphoric acid and acid metal salts such as sodium monohydrogen orthophosphate and potassium hydrogen sulfate. Examples for organic acids are the mono, di and tri carboxylic acids such as acetic, glycolic, lactic, pyruvic, malonic, succinic, glutaric, fumaric, malic, tartaric, citric, ascorbic, maleic, benzoic, phenylacetic, cinnamic, salicylic and sulfonic acids. Salts of the carboxy terminal amino acid moiety include the non-toxic carboxylic acid salts formed with any suitable inorganic or organic bases. These salts include, for example, alkali metals such as sodium and potassium, alkaline earth metals such as calcium and magnesium, and organic primary, secondary and tertiary amines such as trialkylamines.

Typically, the dosage of the GCR fusion protein for the treatment of glycolipid storage disorders like Gaucher's, Tay-Sachs' or Fabry's disease is 0.01 mg to 25 mg, preferably about 0.1 to 2 mg, and more preferably about 0.1 to 1 mg per kilogram body weight per day. The effective dosages may be determined using diagnostic tools which are known in the prior art. In general, the optimum therapeutically acceptable dosage and dose rate for a given patient within the above-said ranges depends on a variety of factors, such as the activity of the specific active material employed, the age, body weight, general health, sex, diet, time and route of administration, rate of clearance or the object of treatment. One skilled in the art will be able to ascertain effective dosages by administration and observing the desired therapeutic effect. The dosages may also vary over the course of therapy, with a relatively high dosage being used initially, until therapeutic benefit is seen, and lower dosages used to maintain the therapeutic benefits.

The invention relates also to therapeutic methods and therapeutic systems for treating a variety of glycolipid storage disorders such as Gaucher's, Fabry's and Tay-Sachs disease and the enzymes related to these diseases, by GCR fusion protein therapy.

The therapeutic method comprises a variety of modalities for practicing the invention in terms of the steps. For example, the GCR fusion protein can be administered following admixture, i.e., simultaneously, or can be administered sequentially with an other drug and/or additive, such as a vitamine or as a single medication. Furthermore, the additives and/or additional drugs and the fusion protein can be separately administered with a time interval between administrations of from zero to 3 weeks, i.e., from substantially immediately after the first active agent is administered to up to 3 weeks after the first agent is administered. Additionally, it is contemplated that the order can be varied, i.e., that the additives and/or additional drugs could be administered prior to administration of the fusion protein, or that administration can be conducted in the reverse order.

In another embodiment, it is considered that the invention can be practiced in conjunction with surgical procedures where for example portions or all of the spleen has been removed. In this regard, the method can be practiced following a surgical procedure. Alternatively, the surgical procedure can be practiced during the interval between administration of the active agent. Exemplary of this method is the combination of the present method with surgical spleen removal.

Treatment according to the method will typically comprise administration of the active agent in one or more cycles of administration. For example, where a single or a simultaneous administration of GCR fusion protein is practiced, a therapeutic composition comprising the single lipid storage disease drug or afore-said drug and a additive and/or another drug is administered over a time period of from about 2 days to about 3 weeks in a single cycle. Thereafter, the treatment cycle can be repeated as needed according to the judgment of the practicing physician. Similarly, where a sequential application of two different agents is contemplated, the administration time will typically cover the same time period. The interval between cycles can vary from about zero to 2 months.

In another embodiment, the invention describes a method for the treatment of Gaucher's disease comprising administering to a patient a therapeutic composition comprising an amount of a GCR fusion protein as defined above as a supportive treatment in combination with a bone marrow transplantation, or a surgery, by removing an organ that serves as an important storage site of glycolipid, for example the spleen or to prepare a successful gene therapy by a previous enzyme augmentation treatment of a human being suffering from a glycolipid storage disorder.

In those cases of a supportive treatment of one of the diseases according to this invention by enzyme replacement or augmentation therapy, the administration of the fusion protein can be separately to the other operation, i.e. the surgery administered with a time interval between the operation and the administrations of the fusion protein of from zero to 3 weeks, i.e., from substantially immediately after the operation, such as bone marrow transplantation, or a surgery of the active agent up to 3 weeks after the agent is administered. Additionally, it is contemplated that the order can be varied, i.e., that the fusion protein could be administered prior to bone marrow transplantation, or a surgery, or that administration can be conducted in the reverse order.

Further, the invention contemplates systems comprising packaging and/or kits which provide the reagents necessary for practicing the methods of the present invention. A kit is therefore described for treating glycolipid storage disorders comprising a package comprising:

a) a therapeutic composition comprising an amount of the GCR fusion protein as defined above

b) optionally a additive or a supportive drug for the treatment of afore-said diseases; and

c) instructions for using the reagents in methods to treat Gaucher's, Tay-Sachs' or Fabry's diseases.

A reagent in a kit of this invention is typically formulated as a therapeutic composition as described herein, and therefore can be in any of a variety of forms suitable for distribution in a kit. Such forms can include a liquid, powder, tablet, suspension and the like formulation for providing the fusion protein of the present invention and optionally the supportive drug and/or additive. The reagents may be provided in separate containers suitable for administration separately according to the present methods, or alternatively may be provided combined in a composition in a single container in the package.

The package may contain an amount sufficient for one or more dosages of reagents according to the treatment methods described herein. Typically, a package will contain an amount sufficient for one cycle of treatment as described herein.

A kit of this invention also contains “instruction for use” of the materials contained in the package. The instructions relate to the use of the fusion protein and/or optionally for the supportive drug and/or the additive for treating the glycolipid storage disorders according to the methods. Insofar as the methods can vary widely depending upon the phase and the type of disease, the patient and the condition of the disease, the instructions can vary to specify procedures for administration accordingly. The invention is not to be considered as limiting as to the nature of the instructions other than the particularity regarding the use of the fusion protein according to the methods of the present invention.

### Sequence Information

The following amino acid sequences were used in this invention

The human lysosomal GCR amino acid sequence (one-letter code)
SEQ ID NO:1
MAGSLTGLLL LQAVSWASGA RPCIPKSFGY SSVVCVCNAT YCDSFDPPTF
PALGTFSRYE STRSGRRMEL SMGPIQANHT GTGLLLTLQP EQKFQKVKGF
GGAMTDAAAL NILALSPPAQ NLLLKSYFSE EGIGYNIIRV PMASCDFSIR
TYTYADTPDD FQLHNFSLPE EDTKLKIPLI HRALQLAQRP VSLLASPWTS
PTWLKTNGAV NGKGSLKGQP GDIYHQTWAR YFVKFLDAYA EHKLQFWAVT
AENEPSAGLL SGYPFQCLGF TPEHQRDFIA RDLGPTLANS THHNVRLLMLD
DQRLLLPHWA KVVLTDPEAA KYVHGIAVHW YLDFLAPAKA TLGETHRLFP
NTMLFASEAC VGSKFWEQSV RLGSWDRGMQ YSHSIITNLL YHVVGWTDWN
LALNPEGGPN WVRNFVDSPI IVDITKDTFY KQPMFYHLGH FSKFIPEGSQ
RVGLVASQKN DLDAVALMHP DGSAVVVVLN RSSKDVPLTI KDPAVGFLET
ISPGYSIHTY LWRRQ
Human IgG1 Fc region-mature protein coding sequence (one-letter code)
SEQ ID NO:2
EPKSCDKTHT CPPCPAPELL GGPSVFLFPP KPKDTLMISR TPEVTCVVVD
VSHEDPEVKF NWYVDGVEVH NAKTKPREEQ YNSTYRVVSV LTVLHQDWLN
GKEYKCKVSN KALPAPIEKT ISKAKGQPRE PQVYTLPPSR EEMTKNQVSL
TCLVKGFYPS DIAVEWESNG QPENNYKTTP PVLDSDGSFF LYSKLTVDKS
RWQQGNVFSC SVMHEALHNH YTQKSLSLSP GK
Human IgG2 Fc region-mature protein coding sequence (one-letter code)
SEQ ID NO:3
ERKCCVECPP CPAPPVAGPS VFLFPPKPKD TLMISRTPEV TCVVVDVSHE
DPEVQFNWYV DGVEVHNAKT KPREEQFNST FRVVSVLTVV HQDWLNGKEY
KCKVSNKGLP APIEKTISKT KGQPREPQVY TLPPSREEMT KNQVSLTCLV
KGFYPSDIAV EWESNGQPEN NYKTTPPMLD SDGSFFLYSK LTVDKSRWQQ
GNVFSCSVMH EALHNHYTQK SLSLSPGK

The following examples describe the invention in more detail without limiting it.

### EXAMPLE 1

### Expression of Human Fc-GCR

A sequence encoding the mature form of GCR was completely synthesized from oligonucleoties by standard techniques.

The synthesized DNA was engineered to have a Xmal-compatible overhang at the 5′ end and an Xhol-compatible overhang at the 3′ end.

The DNA was cloned and sequence analysis confirmed that encodes the mature GCR protein without mutations.

The expression vector pdCs-Fc-GCR was constructed as follows. The Xmal-Xhol-restriction fragment containing the GCR cDNA was ligated to the Xmal-Xhol fragment of the pdCs-Fc vector according to Lo et al. [Protein Engineering (1998) 11:495]. The resultant vector, pdCs-Fc-GCR, was used to transfect mammalian cells for the expression of Fc-GCR. This vector expresses the human imunoglobulin gamma 1 chain Fc-region.

The Fc protein moiety also usually contains a glycosylation site. This site may be optinally changed to a non-glycosylated sequence by standard approaches.

### EXAMPLE 2

### Transfection and Expression of Fc-GCR Fusion Proteins

For transient transfection, the plasmids were introduced into BHK cells. Cells were transfected by coprecipitation of plasmid DNA with calcium phosphate [Sambrook et al. (1989) Molecular Cloning-A Laboratory Manual, Cold Spring, Harbor, N.Y.] or by lipofection using Lipofectamine Plus (Life technologies, Gaithersburg, Md.) according to suppliers protocol.

To generate stable cell lines, NS/0 cells were used for both transient transfection and the generation of stable cel lines.

In order to obtain stably transfected clones, plasmid DNA was introduced into cells by electroporation. About 5×10⁶ cels were washed once with PBS and resuspended in 0.5 ml PBS. 10 μg of linearized plasmid DNA were then incubated with the cells in a Gene Pulser Cuvette (0.4 cm electrode gap, Bio Rad) on ice for 10 min. Electroporation was performed using a Gene Pulser (Bio Rad, Hercules, Calif.) with sttings at 0.25 V and 500 microF. Cells were allowed to recover for 10 min on ice, after which they were resuspended in growth medium and then plated onto 96 well plates. Stably transfected clones were selected by growth in the presence of 100 nM methotrexate (MTX), which was introduced two days post transfection. The cells were fed every 3 days for 2 to 3 more times, and MTX-resistant clones appeared in 2 to 3 weeks. Supernatants from clones were assayed by anti-Fc ELISA to identify high producers. High producing clones were isolated and propadated in growth medium containing 100 nM MTX.

BHK and NS/0 cells were grown in Dulbecco's modified Eagle's medium supplemented with 10% fetal bovine serum, 2 nM glutamine and penicillin/streptomycin.

For routine characterization by gel electrophoresis, Fc fusion proteins in the conditioned media were captured on Protein A Sepharose (Repligen, Cambridge, Mass.) and then eluted by boiling in the protein sample buffer with or without 2-mercaptoethanol. After electrophoresis on a SDS-Gel, the protein bands were visualized by Coomassie staining. For purification, the fusion proteins bound on Protein A Sepharose were eluted in a sodium phosphate buffer (100 mM NaH₂PO₄, pH 3. And 150 mM NaCl). The eluate was then immediately neutralized with 0.1 volume of 2 M Tris-HCl, pH 8.

### EXAMPLE 3

### Carbohydrate Characterization

Endoglycosidase-H was dissolved in 100 mM sodium acetate, pH 6.0, at a final concentration of 10 units/ml. N-glycanase was supplied as a 250 unit/ml suspension in 50% glycerol. Either human placental enzyme or fifty μl aliquot of decyl-agarose fraction containing GCR activity were adjusted to 0.5% SDS/1M β-mercaptoethanol and boiled for two minutes. The samples were then diluted with appropriate buffer to either 200 mM sodium acetate, pH 6.0 (for endoglycosidase-H) or 200 mM sodium phosphate, pH 8.5 (for N-glycanase) to a final composition of 0.1% SDS, 0.7% NP40, and 0.02M β-mercaptoethanol. The samples were again boiled for 1 min and then either endoglycosidase-H or N-glycanase added to final concentrations of 50 mu/ml or 20 U/ml, respectively. Digestions were for about 16 hours at 37° C. Carboxypeptidase Y was used as a control for both deglycosylation reactions.

### EXAMPLE 4

### Amino Acid Sequence Analysis

Samples used for amino acid sequence analysis were electrophoretically fractionated on SDS-Gels as described above and then transferred to PVDF membranes as described by Matsudaira (J.B.C. 262:10035, 1987). Typically, after electrophoresis the gel was incubated in transfer buffer (0.1M CAPS, 10% methanol, pH 11.0) for 10 minutes prior to transblotting (50 ma for 4 hours). The gel was then washed with HPLC grade water for 5 minutes, stained with 0.1% Coomassie Blue R250 (in 50% methanol) for 5 minutes, and finally destained for 10 minutes with 50% methanol-10% acetic acid. The PVDF membrane was again washed with HPLC grade water, dried under a stream of nitrogen and stored in a sealing bag at −20° C. until used for amino acid sequencing.

Amino acid sequence analysis was accomplished using an Applied Biosystems Model 470A gas-phase sequencer equipped with a Model 120A on-line PTH-amino acid analyzer. The program 03R PTH was used directly for sequencing without pretreatment of the membrane strip with polybrene. An approximately 2×8 mm piece of PVDF membrane containing the protein band of interest was excised, centered on the teflon seal, and placed in the cartridge block of the sequencer. Multiple strips of the PVDF membrane could be stacked in this manner, thus increasing the amount of protein available for sequencing. The initial and repetitive yields for sequencing recombinant GCR were calculated by comparison with the yields obtained after 100 picomoles of human placenta GCR were electrophoresed, transblotted to PVDF and subjected to ten cycles of amino acid sequence.

N-terminal amino acid sequence of mature human placental GCR was compared to N-terminal amino acid sequence of recombinant human GCR using the methods described in the text. The N-terminal amino acids determined by direct chemical sequencing of the mature human and recombinant GCR are identical indicating that the signal sequence in the recombinantly produced enzymes are correctly processed.

### EXAMPLE 5

### GCR Assays

For pH profile and inhibition studies, GCR activity was measured using 100 mM potassium phosphate buffer containing 0.15% Triton X-100, 2.5 μl of β-D-1-¹⁴C-glucocerebroside (7.5 mg/ml in sodium taurocholate at 50 mg/ml), and the sample in the total volume of 200 μl. Preincubations with conduritol-B-epoxide were for 30 min at 37° C. For Km determination, β-glucosidase activity was assayed at pH 5.9 using the artificial substrate 4-methylumbellifery-β-D-glucopyranoside (4MUGP) in 100 mM potassium phosphate buffer containing 0.15% Triton X-100 and 0.125% sodium taurocholate. Purification of recombinant GCR was also monitored using 4MUGP.

It is understood that the examples and embodiments described herein are for illustrative purposes only and that various modifications or changes in light thereof will be suggested to persons skilled in the art and are to be included within the spirit and purview of this application and scope of the appended claims.

Other uses will be apparent to one skilled in the art in light of the present disclosures.

1. A fusion protein consisting essentially of an imunoglobuline molecule (Ig) or a fragment thereof and a non immunoglobulin molecule, wherein the non-immunoglobulin molecule is a protein having the biological activity of glucocerebrosidase (GCR-like protein).

2. A fusion protein of claim 1 wherein the Ig molecule has a specificity to a Fc receptor.

3. A fusion protein of claim 1 or 2, wherein the Ig molecule is covalently linked by its C-terminus to the N-terminus of the GCR-like protein.

4. A fusion protein of any of the claims 1 to 3 wherein a linker molecule is fused between the Ig molecule and the GCR-like protein.

5. A fusion protein of claim 4, wherein the linker molecule comprises a protease cleavage site.

6. A fusion protein of claim 5, wherein the protease cleavage site is specific for lysosomal proteases.

7. A fusion protein of any of the claims 1 to 6, wherein the GCR like protein is GCR.

8. A fusion protein of claim 7, wherein GCR is truncated (GCR_{trunc}) or mutated (GCRₘ).

9. A fusion protein of claim 7 or 8, wherein GCR or the GCR-like protein has a modified glycosylation pattern or is non-glycosylated.

10. A fusion protein of any of the claims 1 to 9, wherein the Ig molecule is a Fc portion.

11. A fusion protein of any of the claims 1 to 9, wherein the Ig molecule is a whole antibody.

12. A fusion protein of any of the claims 1 to 11 wherein the Ig molecule or the fragment thereof is designed.

13. A fusion protein of claim 12, wherein the Ig molecule has a reduced affinity to a FcRn receptor.

14. A fusion protein of any of the claims 1 to 13, wherein the Ig molecule within the fusion protein is dimerized.

15. A DNA sequence encoding any of the fusion proteins of claims 1 to 14.

16. A DNA molecule encoding a fusion protein according to at least one of the claims 1 to 14 comprising:
(a) a signal/leader sequence
(b) an Ig molecule
(c) a target protein sequence having the biological activity of GCR.

17. An expression vector comprising a DNA of claim 15 or 16.

18. A host cell suitable for expressing an fusion protein as defined in at least one of the claims 1 to 14 comprising a vector of claim 17.

19. A method for producing a fusion protein of at least one of the claims 1 to 14, said method comprising:
(i) constructing a DNA encoding a precursor protein that comprises a leader sequence for secretion, the Ig molecule, the GCR, GCRₘ or GCR_{trunc} portion and optionally the linker-sequence,
(ii) placing said fused DNA in an appropriate expression vector,
(iii) expressing said fusion protein in a eukaryotic cell, and
(iv) purifying said secreted fusion protein.

20. A pharmaceutical composition comprising a fusion protein according to at least one of the claims 1 to 14 and at least one pharmaceutically acceptable carrier, diluent or excipient.

21. A pharmaceutical composition of claim 20 containing at least one additional pharmaceutically effective drug and/or adjuvants.

22. Use of a fusion protein of any of claims 1 to 14 for the manufacture of a pharmaceutical composition for the treatment of glycolipid storage disorders.

23. The use of claim 22, wherein the glycolipid storage disorder is selectecd from the group consisting of Gaucher's, Fabry's and Tay-Sachs disease.

24. A method of treating glycolipid storage disorders comprising administering to a subject afflicted with said disease a pharmaceutical composition according to claim 16 or 17.

25. The method of claim 18 wherein the glycolipid storage disorder is selectecd from the group consisting of Gaucher's, Fabry's and Tay-Sachs disease.
